(19) United States
(12) Patent Application Publication
(10) Pub. No.: US 2024/0153582 A1
(43) Pub. Date: May 9, 2024

(54) SYSTEMS AND METHODS FOR MYOPIC ESTIMATION OF NUCLEIC ACID BINDING

(71) Applicant: Robert G. Atkinson, Woodinville, WA (US)

(72) Inventor: Robert G. Atkinson, Woodinville, WA (US)

(21) Appl. No.: 17/940,838

(22) Filed: Sep. 8, 2022

Related U.S. Application Data
(60) Provisional application No. 63/242,190, filed on Sep. 9, 2021.

(51) Int. Cl. G16B 30/00

(52) U.S. Cl. CPC G16B 30/00

(57) ABSTRACT

Systems and methods for capturing desired interactions and non-interactions of a set of strands (e.g., DNA strands, RNA strands) that may be used in various technological applications, including DNA molecular probes used in medical diagnostics, forensics, microbial ecology, molecular computation, DNA origami, and numerous other applications. Given a nucleic acid system with a certain defined binding pattern, the implementations of the present disclosure automatically generate nucleotide sequences that achieve the intended binding pattern and not other binding patterns, subject to miscellaneous other constraints of the system. Advantageously, given nucleotides i and j, the implementations of the present disclosure consider the binding behavior only in a limited neighborhood of nucleotides surrounding each nucleotide in their respective strands, rather than the binding behavior of whole assignment of which they are a part. These features provide a time-efficient and incremental algorithm that is suitable for numerous practical applications.

### REFERENCE TO AN ELECTRONIC SEQUENCE LISTING

The contents of the electronic sequence listing (120342 402 SEQUENCE LISTING.xml; Size: 21252 bytes; and Date of Creation: Jun. 9, 2023) is herein incorporated by reference in its entirety.

### BACKGROUND

### Technical Field

The present disclosure generally relates systems and methods of nucleic acid sequence design and applications.

### Description of the Related Art

Synthetic nucleic acid design involves the process of generating a set of nucleic acid base sequences that will associate or assemble into a desired conformation. Nucleic acid design is used the fields of DNA nanotechnology, DNA computing and other fields. Nucleic acid design is necessary because there are many possible sequences of nucleic acid strands that will fold into a given secondary structure, but many of these sequences will have undesired additional interactions which should be avoided. Further, there are many tertiary structure considerations which may affect the choice of a secondary structure for a given design. Nucleic acid design can be considered the inverse of nucleic acid structure prediction. In structure prediction, the structure is determined from a known sequence, while in nucleic acid design, a sequence is generated which will form a desired structure.

The structure of nucleic acids includes a sequence of nucleotides. Generally, there are four types of nucleotides distinguished by which of the four nucleobases they contain. In DNA, these types are adenine (A), cytosine (C), guanine (G), and thymine (T). In RNA, these are A, C, G, and uracil (U). Nucleic acids have the property that two molecules will bind to each other to form a double helix only if the two sequences are complementary, that is, they can form matching sequences of base pairs. Thus, in nucleic acids the sequence determines the pattern of binding and thus the overall structure.

Nucleic acid design is the process by which, given a desired target structure or functionality, sequences are designed and generated for nucleic acid strands which will self-assemble into that target structure. Nucleic acid design may encompass multiple levels of nucleic acid structure, including primary structure, secondary structure, and tertiary structure. Generally, primary structure is the raw sequence of nucleobases of each of the component nucleic acid strands; secondary structure is the set of interactions between bases, i.e., which parts of which strands are bound to each other; and tertiary structure is the locations of the atoms in three-dimensional space, taking into consideration geometrical and steric constraints.

A primary concern in nucleic acid design is ensuring that the target structure has the lowest free energy (i.e., is the most thermodynamically favorable) whereas misformed structures have higher values of free energy and are thus unfavored. These goals can be achieved through the use of a number of approaches, including heuristic, thermodynamic, and geometrical approaches, and combinations thereof. Two considerations in nucleic acid design are that desired hybridizations should have melting temperatures in a narrow range, and any spurious interactions should have very low melting temperatures (i.e., they should be very weak). There is also a contrast between affinity-optimizing “positive design,” which seeks to minimize the energy of the desired structure in an absolute sense, and specificity-optimizing “negative design,” which considers the energy of the target structure relative to those of undesired structures. Algorithms which implement both kinds of design tend to perform better than those that consider only one type.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

In the drawings, identical reference numbers identify similar elements or acts. The sizes and relative positions of elements in the drawings are not necessarily drawn to scale.

For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 shows an example workflow for a programming tool for engineering DNA oligonucleotides (“oligos”), according to one illustrated implementation.

FIGS. 2A-2C are an illustration of a plurality of nucleotide bases of a design example for LAMP internal amplification control (IAC). FIGS. 2A-2C contain SEQ ID NOS: 1-20.

FIG. 3 is a graph showing the binding intent for the LAMP IAC design example.

FIGS. 4A-4B show an example scoring matrix for the LAMP IAC design example.

FIG. 5 is an illustration of example computing binding probabilities for a system of two 20 mers (“forward” and “reverse”) and their complements.

FIG. 6 shows a simplified example matrix that includes elements p_{i,j} that are the precomputed binding probabilities for nucleotides i and j, which probabilities consider the binding behavior only in a limited myopic neighborhood surrounding each nucleotide i and j in their respective strands. FIG. 6 contains SEQ ID NOS: 21-22.

FIG. 7 shows an illustration of a pair-wise central binding probability for T and A in the strands actTttt and taaActc, respectively, obtained using an analysis tool.

FIG. 8A shows a table of the number of database entries and database size (in MB) for neighborhood sizes between 1 and 10 nucleotides.

FIG. 8B shows a graph of the size of a database storing binding probabilities as a function of neighborhood size.

FIG. 9 is a flowchart for a method of performing synthetic nucleic acid sequence design, according to one non-limiting illustrated implementation.

FIG. 10 is a flowchart for a method of evaluating a cost function that may be performed as part of the method of FIG. 9, according to one non-limiting illustrated implementation.

FIG. 11 is a flowchart for a method of mutating nucleotides, re-scoring, and looping until a threshold is satisfied, which method may be performed as part of the method of FIG. 9, according to one non-limiting illustrated implementation.

FIG. 12 is a block diagram showing some of the components typically incorporated in at least some of the computer systems and other devices on which the implementations of the present disclosure may operate.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts).

Reference throughout this specification to “one implementation” or “an implementation” means that a particular feature, structure or characteristic described in connection with the implementation is included in at least one implementation. Thus, the appearances of the phrases “in one implementation” or “in an implementation” in various places throughout this specification are not necessarily all referring to the same implementation.

Furthermore, the particular features, structures, or characteristics may be combined in any suitable manner in one or more implementations.

As used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The headings and Abstract of the Disclosure provided herein are for convenience only and do not interpret the scope or meaning of the implementations.

One or more implementations of the present disclosure are directed to unique computing systems and methods that allow for the determination of the desired interactions and non-interactions of a set of strands (e.g., DNA strands, RNA strands) that may be used in various technological applications, including DNA molecular probes used in medical diagnostics, forensics, microbial ecology, molecular computation, DNA origami, and numerous other applications. In at least some implementations, the problem may be stated as follows: given a nucleic acid system with a certain defined binding pattern, manifest nucleotide sequences that achieve the intended binding pattern and not other binding patterns, subject to miscellaneous other constraints of the system. The search space for this problem is incomprehensibly large, for example, 4ⁿ for sequences of length n. Thus, an exhaustive search is impossible. In at least some implementations, the problem may be stated as, given an assignment A of nucleotides to the strands of a particular DNA system design, a goal of the DNA design algorithms of the present disclosure is to evaluate a scoring function for that assignment. This may be a cost function wherein lower is better, with the best possible score being zero, for example.

The cost function should mirror physical reality. In other words, a cost function with a higher value should be more likely to produce nucleotide binding patterns that are further from those intended by the design. Conversely, a cost function near zero should be more likely to produce nucleotide binding patterns that achieve the intended result. The cost function need not model the underlying physics; it can instead heuristically approximate it, though to the extent that the heuristic departs from reality results are likely to degrade.

The cost function may be used in the context of a global optimization algorithm such as iterated local search or simulated annealing. Briefly, in most such algorithms, a random legal assignment of nucleotides is performed, and the cost is evaluated. Then, some random subset of the nucleotides (typically a handful) are mutated to different legal assignments, and the cost is re-evaluated. If the cost improves, the mutations are retained. If the cost does not improve, the mutations are (likely) rejected. Experience shows that millions of iterations are necessary to achieve intended binding patterns in most designs.

Before discussing implementations of the present disclosure, a naive cost function that works, but is unusable, is first examined. We consider the interaction of every nucleotide i in every strand in the design against every nucleotide j in every strand in the design. The Dirks-Pierce algorithm gives the probability p_{i,j} that nucleotides i and j will pair in equilibrium. See Dirks, R. M. and N. A. Pierce (2004). “An algorithm for computing nucleic acid base-pairing probabilities including pseudoknots.” J Comput Chem 25(10): 1295-1304. It is noted that this binding probability is independent of concentration, but is dependent on temperature and free energy (e.g., salts, etc.). The design of the DNA system on the other hand, gives us an intention, t_{i,j}, as to whether these nucleotides i and j are intended to pair or not. As an example, t_{i,j} may be equal to 1 for an intended pairing and −1 for an intended non-pairing.

In at least some implementations, a pair-wise cost, c_{i,j}, associated with nucleotides i and j, may be calculated according to the following function:

c_{i,j}=(t_{i,j}+1)/2−(t_{i,j}×p_{i,j})

It is noted that the above formula is a conditional-free form of the function that yields 1−p_{i,j} for an intended pairing and p_{i,j} for an intended non-pairing.

In other implementations, the cost function c_{i,j} may be zero if the pairing is intended and p_{i,j} for an intended non-pairing. This second example cost function may be advantageous because it may be more worthwhile to penalize an unintended pairing rather than reward an intended pairing.

An overall cost for the assignment A as a whole can then be assessed by summing the pair-wise costs over all i and j. This cost may then be used in global optimization, as discussed above.

While the above-discussed algorithm and cost function are viable in theory, in practice they are unusable, as evaluating an algorithm (e.g., Dirks-Pierce algorithm) on every mutation cycle is a prohibitive time expense due to the enormous search space and the time required to evaluate the algorithm on every mutation cycle.

As an improved approach, an approximation to the naive function discussed above may be used. The key idea is that given nucleotides i and j, we consider the binding behavior only in a limited neighborhood surrounding each nucleotide in their respective strands, rather than the binding behavior of whole assignment A of which they are a part. The inventor of the present disclosure has found that it is the neighborhood of nucleotides that has the most influence of the binding behavior.

The neighborhood around the nucleotide i is the k nucleotides in the nucleotide i's strand for which i is the middle, wherein k is a relatively small integer (e.g., 3, 7, 11, 16). The neighborhood around the nucleotide j is the analogous k nucleotides around the nucleotide j in the nucleotide j's strand. With these neighborhoods, the method retrieves in precomputed storage the binding probability p_{i,j} which has been generated by running an algorithm (e.g., Dirks-Pierce algorithm, etc.) on a system that includes only these two contextual k-length strands as input. We then proceed as discussed above with iteratively mutating nucleotides, evaluating the cost function, and continuing until a threshold condition is reached (e.g., progress has stalled, number iterations, elapsed time).

This algorithm is time-efficient. The evaluation of an algorithm (e.g., Dirks-Pierce algorithm) on each mutational cycle has been replaced with a simple lookup in memory. It is further incremental, in that mutations will only affect the costs of and require the re-evaluation of the nucleotide pairs (i,j) in the immediate vicinity of the mutation. That is, distant pairs are unaffected, and can retain scores from the previous mutational cycle. If there are n nucleotides in the design, each cycle thus needs approximately 2 kn evaluations (specifically, 2 kn−k²). That is, the cost function has time-expense linear in the size of the problem input; it is O(n).

The neighborhood surrounding a nucleotide i may include at most k symbols. If the nucleotide i is in the middle of a long strand, then all of those symbols are nucleotide bases. However, if the nucleotide i is near the end of its strand, some of those symbols may be blanks. Using the Java regular expression language, the general structure of the neighborhood at the nucleotide i is:

(\s)*[agct]+(\s)*

Further, the number of blanks on either side of the nucleotide i should be balanced such that there is always a nucleotide (not a blank) in the middle of the neighborhood.

In order to successfully work with neighborhoods, pieces of functional infrastructure were developed, and are discussed below for explanatory purposes.

First, it was determined how many neighborhoods of a given length there are there. The variable k is the size of the neighborhood, as noted above, and the variable b is the size of the nucleotide alphabet. Usually the variable b is four (e.g., A, C, G, T), but the variable b can be smaller or larger if non-traditional bases are permitted.

A number of Mathematica® utility functions were developed to implement the algorithms of the present disclosure. These functions are discussed below, with non-limiting examples of code presented in-line. The function show[ ] is used to evaluate and exhibit test results, while the function stringTake[ ] provides a simple generalization of the built-in StringTake[ ] function.

Clear[show]
show[x_] := Inactivate[x] → x
SetAttributes[show,| HoldAll]
show[Factorial[7]]
Clear[stringTake]
stringTake[str_, 0] := “”
stringTake[str_, val_] := StringTake[str, val]
7: → 5040

The function numStrands[ ] gives the number of strands of a given length for an alphabet of a given size. Note that there is exactly one zero-length strand: “ ”.

Clear [numStrands]
numStrands[k_, b_] := b\hat{ }k
# → numStrands [#, 4] & /@ Range [0, 10]
{0 → 1, 1 → 4, 2 → 16, 3 → 64, 4 → 256, 5 → 1024,
6 → 4096, 7 → 16 384, 8 → 65 536, 9 → 262 144, 10 → 1 048 576}

The function numStrandsAtMost[ ] does the same, but for a given maximum size rather than a fixed size.

Clear [numStrandsAtMost]
numStrandsAtMost [k_, b_] := Sum [numStrands [kk, b], {kk, 0, k})
numStrandsAtMost [k, b]
# → numStrandsAtMost [#, 4] & /@ Range [0, 10]
$\frac{- 1 + {b}^{1 + k}}{- 1 + b}$
{0 → 1, 1 → 5, 2 → 21, 3 → 85, 4 → 341, 5 → 1365,
6 → 5461, 7 → 21 845, 8 → 87 381, 9 → 349 525, 10 → 1 398 101}

It is noted that for the function numStrandsAtMost[ ], the sum can be expressed in closed form. Consider k odd, wherein k=2n+1. Then, any given neighborhood comprises: (1) a central, focus nucleotide; (2) a sequence of nucleotides of length 0 . . . n to the left, followed (reading right to left), possibly, by blanks; and (3) a sequence of nucleotides of length 0 . . . n to the right, followed (reading left to right), possibly, by blanks. If k is even, k=2n, then there are two cases: the focus nucleotide is either just-left or just-right of center. In each case we have: (1) the focus nucleotide; (2) a sequence of nucleotides of length 0 . . . n on one side, followed possibly by blanks; and (3) a sequence of nucleotides of length 0 . . . n-1 on the other side, followed possibly by blanks.

Without loss of generality, in the ordinal work below we assume that the shorter of these two sequences (of the even case) is always on the left (5′) side of the focus nucleotide. In the code, this will be the “ordinal” focus of the neighborhood.

In sum, the number of neighborhoods of length k can be computed as follows:

```
Clear [numHoods, numHoodsOdd, numHoodsEven]
numHoodsOdd[n_, b_] := numStrandsAtMost[n, b] * b * numStrandsAtMost[n, b]
numHoodsEven[0, b_] := 1 (* not a real neighborhood,
but definition helps in recursions *)
numHoodsEven[n_, b_] := numStrandsAtMost[n, b] * b * numStrandsAtMost[n - 1, b]
numHoods[k_, b_] := Piecewise [{
    {numHoodsOdd[(k - 1) /2, b], OddQ [k]},
    {numHoodsEven[k / 2, b], EvenQ [k] 1),
  0]
numHoodsOdd[n, b] / / show
numHoodsEven[n, b] / / show
# → numHoods[#, 4] & /@ Range [0, 10]
$numHoodsOdd [ n , b ] \to \frac{{b ( - 1 + {b}^{1 + n} )}^{2}}{{( - 1 + b )}^{2}}$
$numHoodsEven [ n , b ] \to \frac{b ( - 1 + {b}^{1 + n} ) ( - 1 + {b}^{1 + n} )}{{( - 1 + b )}^{2}}$
{0 → 1, 1 → 4, 2 → 20, 3 → 100, 4 → 420, 5 → 1764,
6 → 7140, 7 → 28 900, 8 → 115 940, 9 → 465 124, 10 → 1861 860}
```

When contemplating the interaction of two arbitrary nucleotides i and j, we have, generally, two arbitrary at-most-k-sized neighborhoods surrounding each nucleotide. Thus we want to know about the number of different neighborhood pairs that exist. Conceptually, as each neighborhood can pair with any other of the same length, naively there are numHoods[k,b]×numHoods[k,b] neighborhood pairs. We can think of this as a numHoods[k,b]×numHoods[k,b] matrix, where any given cell represents the interaction of two particular neighborhoods. However, there are redundancies and nuances to take into account.

Specifically, for k odd, each neighborhood pair has the focus nucleotide exactly in the center. There is thus a two-fold 180° rotational symmetry of the double stranded DNA formed by the binding of the two neighborhood strands (two-fold, excepting self-symmetric dsDNA). In detail, what we need is the lower triangular part of the matrix, including the whole diagonal. That's the sum of 1 . . . numHoodsOdd[n,b].

Fork even, one neighborhood of the pair will have the focus nucleotide just left of center while the other will have it just right of center. Thus, there is no rotational symmetry, and we need the whole matrix.

```
Clear [numHoodPairs, numHoodPairsOdd, numHoodPairsEven]
numHoodPairsOdd[n_, b_] := Sum[i, {i, 1, numHoodsOdd[n, b]}]
numHoodPairsEven[n_, b_] := numHoodsEven[n, b] * numHoodsEven[n, b]
numHoodPairs[k_, b_] := Piecewise[{
numHoodsEven[n_, b_] := numStrandsAtMost[n, b] * b * numStrandsAtMost[n - 1, b]
numHoods[k_, b_] := Piecewise [{
    {numHoodPairsOdd[(k - 1) / 2, b], OddQ[k]},
    {numHoodPairsEven[k / (2, b], EvenQ[k]}},
  0]
numHoodPairsOdd[n, b] / / show
numHoodPairsEven[n, b] / / show
# → numHoodPairs[#, 4] & /@ Range [0, 10]
$numHoodPairsOdd [ n , b ] \to \frac{{b ( - 1 + {b}^{1 + n} )}^{2} ( 1 - b + {b}^{2} - 2 {b}^{2 + n} + {b}^{3 + 2 n} )}{2 {( - 1 + b )}^{4}}$
$numHoodPairsEven [ n , b ] \to \frac{{{b}^{2} ( - 1 + {b}^{n} )}^{2} {( - 1 + {b}^{1 + n} )}^{2}}{{( - 1 + b )}^{4}}$
{0 → 1, 1 → 10, 2 → 400, 3 → 5050, 4 → 176 400, 5 → 1 556 730, 6 → 50 979 600,
7 → 417 619 450, 8 → 13 442 083 600, 9 → 108 170 400 250, 10 → 3 466 522 659 600
```

For each pair of neighborhoods, we need to store the probability that the focus nucleotides i and j will bind, given the neighborhoods. Let the variable f be the number of bytes used to store this value in memory. In practice, it is likely that f will be four, reflecting the use of single-precision floating point numbers, but other values may be used dependent on the implementation.

```
Clear[sizeNeeded, sizeNeededEven, sizeNeededOdd]
sizeNeededOdd[n_, b_, f_] := numHoodPairsOdd[n, b] * f
sizeNeededEven[n_ b_ f_] := numHoodPairsEven[n, b] * f
sizeNeeded[k_, b_, f_] := Piecewise [{
    {sizeNeededOdd[(k - 1) / 2, b], OddQ[k]},
    {sizeNeededEven[k / 2, b], EvenQ[k]}},
  0]
sizeNeededOdd[n, b, f] / / show
sizeNeededEven[n, b, f] / / show
# → sizeNeeded[#, 4, 4] & /@ Range[0, 10]
# → StringJoin[ToString[sizeNeeded[#, 4, 4] * 1.*\hat{ }-6, TraditionalForm],
″ MB″] & /@ Range[1, 10]
$sizeNeededOdd [ n , b , f ] \to \frac{{b ( - 1 + {b}^{1 + n} )}^{2} ( 1 - b + {b}^{2} - 2 {b}^{2 + n} + {b}^{3 + 2 n} ) f}{2 {( - 1 + b )}^{4}}$
$sizeNeededEven [ n , b , f ] \to \frac{{{b}^{2} ( - 1 + {b}^{n} )}^{2} {( - 1 + {b}^{1 + n} )}^{2} f}{{( - 1 + b )}^{4}}$
{1 → 40, 2 → 1600, 3 → 20 200, 4 → 705 600, 5 → 6 226 920, 6 → 203 918 400,
7 → 1 670 477 800, 8 → 53 768 334 400, 9 → 432 681 601 000, 10 → 13 866 090 638 400}
{1 → 0.00004 MB, 2 → 0.0016 MB, 3 → 0.0202 MB, 4 → 0.7056 MB, 5 → 6.22692 MB,
6 → 203.918 MB, 7 → 1670.48 MB, 8 → 53768.3 MB, 9 → 432682. MB, 10 → 1.38661 × 10⁷ MB}
```

As indicated above, the storage requirements grow quickly as k is increased. In view of the storage costs, one can conclude that k=7 may be an advantageous number, as the entire state (totaling 1,670.48 MB) can reside in RAM on a reasonable machine. The state for k=9, totaling 432,682 MB, would fit on a moderately sized solid-state drive (SSD), and may also be advantageous in certain implementations.

A function is then determined that, given the ordinals of a pair of neighborhoods, yields the integer index into a database where we will find the data associated with that neighborhood pair. This is a two-dimensional indexing problem. Thus, the central problem is defining a total order over the set of neighborhoods of a given length. With that in hand, a two-dimensional matrix indexing approach (for the k even case) or lower-triangular-matrix indexing approach (for the k odd case) may be used to convert the pair of ordinals into the linear index for the database.

Our approach is to define an ordinal function for strands of a given length, then one for strands at most a certain length, and from that construct an ordinal function for neighborhoods. We define a total ordering over strands of a given, fixed length. The approach is to treat strands as “base-b” integers (e.g., base-4 integers), with the nucleotide letters representing the digits 0 . . . b-1 (e.g., 0 . . . 3) in a defined order. The function ordinalStrandSameLength[ ] returns the (zero-based) ordinal of a strand within all strands of exactly the same length.

```
Clear[ordinalStrandSameLength, digitOfNucleotide]
digitOfNucleotide [nucleotide_, letters_] := Module[
    {rgchLetters = Characters [letters]},
    Position [rgchLetters, nucleotide] [[1, 1]] − 1
  ];
ordinalStrandSameLength [strand_String, letters_String] := Module[
    {rgchStrand = Characters [strand], base, digits},
    base = StringLength [letters];
    digits = digitOfNucleotide [#, letters] & /@ rgchStrand;
    FromDigits [digits, base]
  ]
ordinalStrandSameLength [“”, “agct”] // show
ordinalStrandSameLength [“a”, “agct”] // show
ordinalStrandSameLength [“t”, “agct”] // show
ordinalStrandSameLength [“ta”, “agct”] // show
ordinalStrandSameLength [“taa”, “agct”] // show
ordinalStrandSameLength [“tcggtat”, “agct”] // show
ordinalStrandSameLength [“ttttttt”, “agct”] // show
ordinalStrandSameLength [, agct] → 0
ordinalStrandSameLength [a, agct] → 0
ordinalStrandSameLength [t, agct] → 3
ordinalStrandSameLength [ta, agct] → 12
ordinalStrandSameLength [taa, agct] → 48
ordinalStrandSameLength [tcggtat, agct] → 14 707
ordinalStrandSameLength [ttttttt, agct] → 16 383
```

The function ordinalFromStrand[ ] returns the ordinal of a strand within all strands of any length. The function ordinalStrandMax[ ] returns one more than the last valid ordinal for strands of a given length. That should be the same as numStrandsAtMost[ ].

```
Clear[ordinalFromStrand, ordinalFromStrand$, ordinalStrandMax]
ordinalFromStrand[strandData_String, letters_] :=
  ordinalFromStrand$[strand[strandData, StringLength[strandData]],
  letters]
ordinalFromStrand$[strand[strandData_, 0], letters_] :=
  ordinalStrandSameLength[strandData, letters]
ordinalFromStrand$[strand[strandData_, length_], letters_] := Module[
      { }
      numStrandsAtMost[length − 1, StringLength[letters]] +
        ordinalStrandSameLength[strandData, letters]
    ]
ordinalStrandMax[0_, letters_] := ordinalFromStrand[“”, letters] + 1
ordinalStrandMax[len_, letters_] := Module[
      {digit},
      digit = stringTake[letters, −1];
      ordinalFromStrand[StringRepeat[digit, len], letters] + 1
    ]
ordinalFromStrand[“”, “agct”] // show
ordinalFromStrand[“a”, “agct”] // show
ordinalFromStrand [“t”, “agct”] // show
ordinalFromStrand [“aa”, “agct”] // show
ordinalFromStrand [“taaa”, “agct”] // show
ordinalFromStrand [“taaaa”, “agct”] // show
ordinalFromStrand [“taaaaa”, “agct”] // show
ordinalFromStrand [“ttc”, “agct”] // show
# → numStrandsAtMost[#, 4] & /@ Range [0, 10]
# → ordinalStrandMax[#, “agct”] & /@ Range[0, 10]
ordinalFromStrand [, agct] → 0
ordinalFromStrand [a, agct] → 1
ordinalFromStrand [t, agct] → 4
ordinalFromStrand [aa, agct] → 5
ordinalFromStrand [taaa, agct] → 277
ordinalFromStrand [taaaa, agct] → 1109
ordinalFromStrand [taaaaa, agct] → 4437
ordinalFromStrand [ttc, agct] → 83
{0 → 1, 1 → 5, 2 → 21, 3 → 85, 4 → 341, 5 → 1365,
6 → 5461, 7 → 21 845, 8 → 87 381, 9 → 349 525, 10 → 1 398 101}
{0 → 1, 1 → 5, 2 → 21, 3 → 85, 4 → 341, 5 → 1365,
6 → 5461, 7 → 21 845, 8 → 87 381, 9 → 349 525, 10 → 1 398 101}
```

The next step is to determine neighborhood ordinals. The function cleanNeighborhood[ ] replaces all non-nucleotide letters in a neighborhood with blanks, yielding a canonical representation. The function wellFormedNeighborhood[ ], when invoked on a cleaned neighborhood, indicates whether the neighborhood is structurally sound, i.e., there are spaces only on either side, not in the middle of the neighborhood. The function splitNeighborhood[ ] takes a legal (cleaned) neighborhood, and splits it into its adjoining spaces and central nucleotides. It is noted that there are length requirements on the adjoining spaces that go beyond what is tested for in well-formedness.

```
Clear[cleanNeighborhood]
cleanNeighborhood[hood_, letters_] :=
  Module[{rgchNeighborhood = Characters[hood], rgchLetters = Characters[letters]},
    If[Length[Position[rgchLetters, #]] > 0, #, “ ”] &/@ rgchNeighborhood // StringJoin
  ]
cleanNeighborhood[“...acgtc.”, “agct”] // show
Clear[wellFormedNeighborhood]
wellFormedNeighborhood[hood] :=
  StringMatchQ[hood, RegularExpression[“\hat{ }\\s*[\hat{ }\\s]+\\s*$”]]
wellFormedNeighborhood[cleanNeighborhood[“...acgtc.”, “agct”]] // show
wellFormedNeighborhood[cleanNeighborhood[“...ac tc.”, “agct”]] // show
Clear[splitNeighborhood]
splitNeighborhood[legalNeighborhood_] := StringCases[legalNeighborhood,
  RegularExpression[“\hat{ }(\\s*)([\hat{ }\\s]+)(\\s*)$] :> Sequencer[“$1”, “$2”, “$3”]]
splitNeighborhood[“ aaa ”] // show
splitNeighborhood[“ a a ”] // show
cleanNeighborhood[...acgtc., agct] → acgtc
wellFormedNeighborhood[cleanNeighborhood[...acgtc., agct]] → True
wellFormedNeighborhood[cleanNeighborhood[...ac tc., agct]] → False
splitNeighborhood[ aaa ] → {, aaa, }
splitNeighborhood[ a a ] → { }
```

We have three representations of a neighborhood, exemplified as follows. Representation A is a natural one for input, while representation C is a natural for computing. Representation B is a transitional state between the two. This is illustrated by the following example:

```
a → hood[“ act ”];
b →hood[3, {“act”}, 1];
c → hood[{“”, “a”, “ct”}, 7];
Clear[toNeighborhoodB, toNeighborhoodC]
toNeighborhoodB[hood[data_String], letters_String] := Module[
    {clean = cleanNeighborhood[data, letters], split, k, 1b, 1, r, rb,
    ifocus}, split = splitNeighborhood[clean];
    1b = StringLength[split[1]]];
    rb = StringLength[split[3]]];
    hood[1b, {split[2]]}, rb]
  ]
toNeighborhoodC[hood[1b_, {nts_}, rb_], letters_] := Module[
    {k, ifocus, 1, r, left, right, focus},
    k = 1b + StringLength[nts] + rb;
    ifocus = Ceiling[k/2];
    1 = ifocus − 1b − 1;
    r = k − ifocus − rb;
    left = stringTake[nts, 1];
    right = stringTake[nts, −r];
    focus = stringTake[nts, {ifocus − 1b}];
    hood [{left, focus, right}, k]
  ]
toNeighborhoodC[c: hood[data_String], letters_String] :=
toNeighborhoodC[toNeighborhoodB[c, letters], letters]
toNeighborhoodB[hood[“...atg.”], “agct”] // show
toNeighborhoodC[hood[“...atg.”], “agct”] // show
toNeighborhoodB[hood[“a.”], “agct”] // show
toNeighborhoodC[hood[“a.”], “agct”] // show
toNeighborhoodB[hood[...atg.], agct] → hood[3, {atg}, 1]
toNeighborhoodC[hood[...atg.], agct] → hood[{, a, tg}, 7]
toNeighborhoodB[hood[a.], agct] → hood [0, {a}, 1]
toNeighborhoodC[hood[a.], agct] → hood [{, a, }, 2]
```

As discussed above, a neighborhood is characterized by a focus nucleotide in the middle (a one-nucleotide sequence) and “at most” wing sequences to the left and right.

From the above, we generate ordinals for each of those three sequences within their respective spaces. We can use the same to construct a three “digit” neighborhood ordinal, where the corresponding numeric base is appropriately different for each such digit (see by way of comparison the Mathematica function MixedRadix[ ]).

The numeric base for the focus nucleotide is (usually) four. For k odd, the base for the left and right wing sequences is numStrandsAtMost[(k−1)/2]; for k even, the left wing has base numStrandsAtMost[k/2−1] and the right wing numStrandsAtMost[k/2]. Put differently, that is Floor[(k+1)/2] in all cases.

We could reasonably order the three “digits” of a neighborhood in whatever order we wished. The only important criterion for selecting one over another seems to be cache locality. But so far as can be discerned, the access pattern will be pretty random, so may not be a pragmatic concern. Our arbitrary choice is {left, right, focus}.

```
Clear[ordinalFromNeighborhood]
ordinalFromNeighborhood[hood[{left_, focus_, right_}, 0], letters_String] := 0
ordinalFromNeighborhood[
    hood[{left_, focus_, right_}, k_]., letters_String] := Module[
    {lettersAndBlank, digits, bases, kLeft, kRight},
    lettersAndBlank = “ ” <> letters;
    kLeft = Floor[(k−1)/2];
    kRight = Floor[k/2];
    digits = {ordinalFromStrand[left, letters],
      ordinalFromStrand[focus, letters), ordinalFromStrand[right, letters]};
    bases = {ordinalStrandMax[kLeft, letters], ordinalStrandMax[1, letters],
      ordinalStrandMax[kRight, letters]};
    digits = digits[{1, 3, 2}]];
    bases = bases[{1, 3, 2}]];
    {digits, bases, FromDigits(digits, MixedRadix[bases]));
    FromDigits[digits, MixedRadix[bases]]
  ]
ordinalFromNeighborhood[c: hood[data_], letters_] :=
  ordinalFromNeighborhood[toNeighborhoodC[c, letters], letters]
ordinalFromNeighborhood[c: hood[data_]] := ordinalFromNeighborhood[c, “agct”]
ordinalFromNeighborhood[hood[“tttta..”]] // show
ordinalFromNeighborhood[hood[“a”] // show
ordinalFromNeighborhood[hood[“t”]] // show
ordinalFromNeighborhood[hood[“a.”]] // show
ordinalFromNeighborhood[hood[“.a.”]] // show
ordinalFromNeighborhood[hood[tttta..]] → 35 709
ordinalFromNeighborhood[hood[a]] → 1
ordinalFromNeighborhood[hood[t]] → 4
ordinalFromNeighborhood[hood[a.]] → 1
ordinalFromNeighborhood[hood [.a]] → 1
```

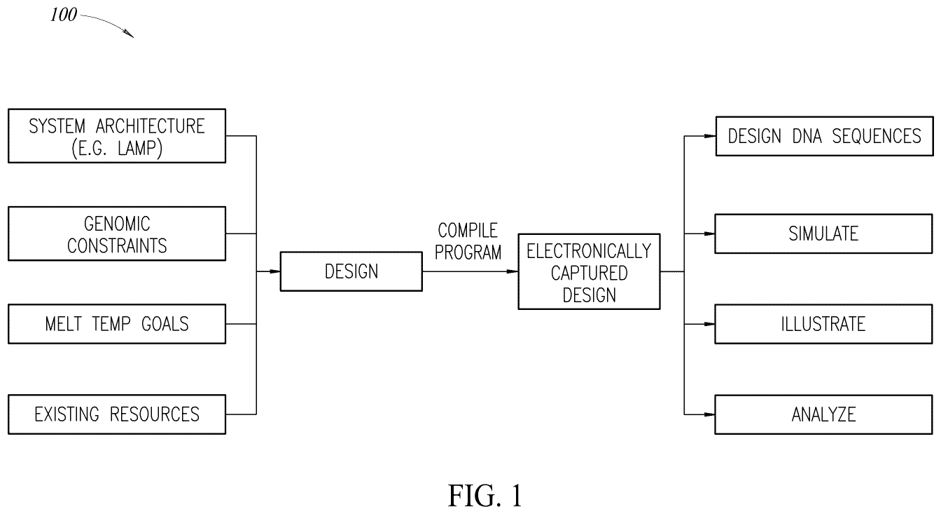

FIG. 1 shows an example workflow 100 for a programming tool for engineering DNA oligonucleotides (“oligos”). Example inputs may include system architecture (e.g., loop-mediated isothermal amplification (LAMP)), genomic constraints, melt temperature goals and existing resources, that are all compiled into a program. Once the design is electronically captured, the workflow may include designing, simulating, illustrating, and analyzing DNA sequences. The output of the compilation may include, for example, (1) a set of strands each of a fixed length; (2) for every pair of nucleotides in every pair of strands (a large matrix), binding intent and a weighted importance of correct binding or non-binding; (3) for every nucleotide in every strand, a probability sampling distribution (e.g., fixed, three-letter alphabet, etc.); (4) optionally, a melt temperature goal for each strand; and (5) optionally, pattern matching constraints for each strand and domain.

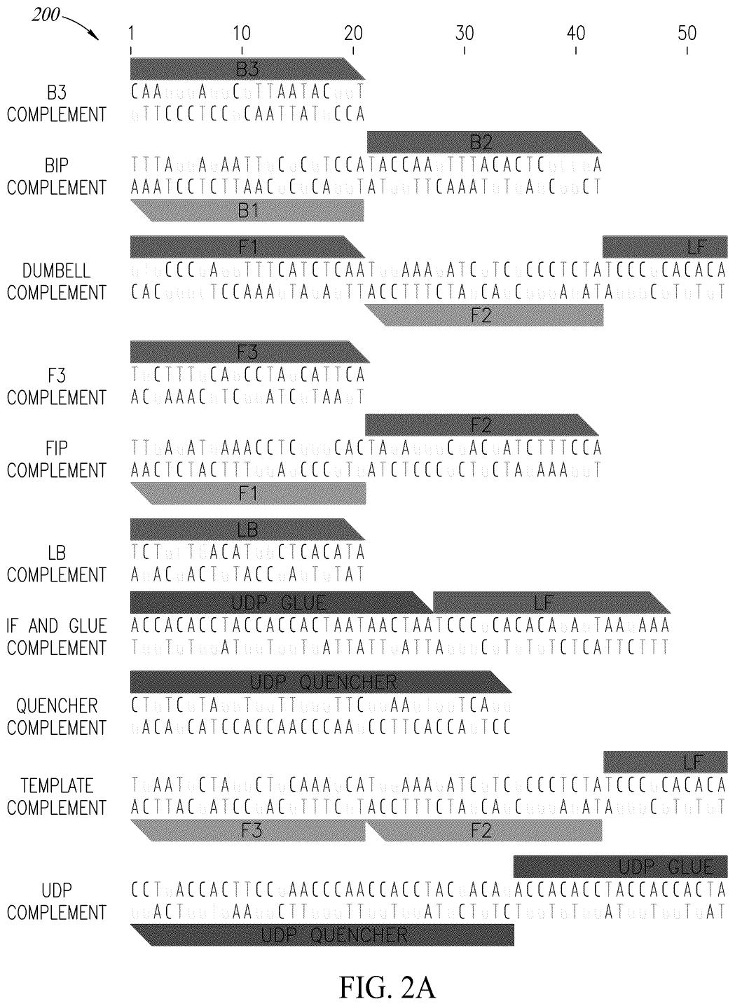

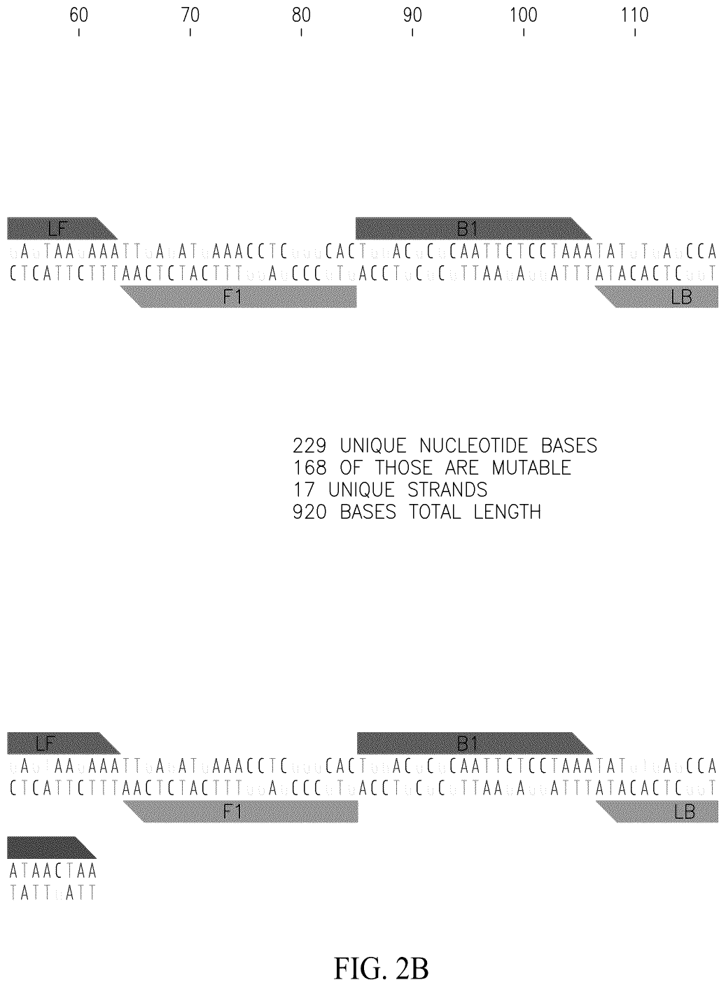

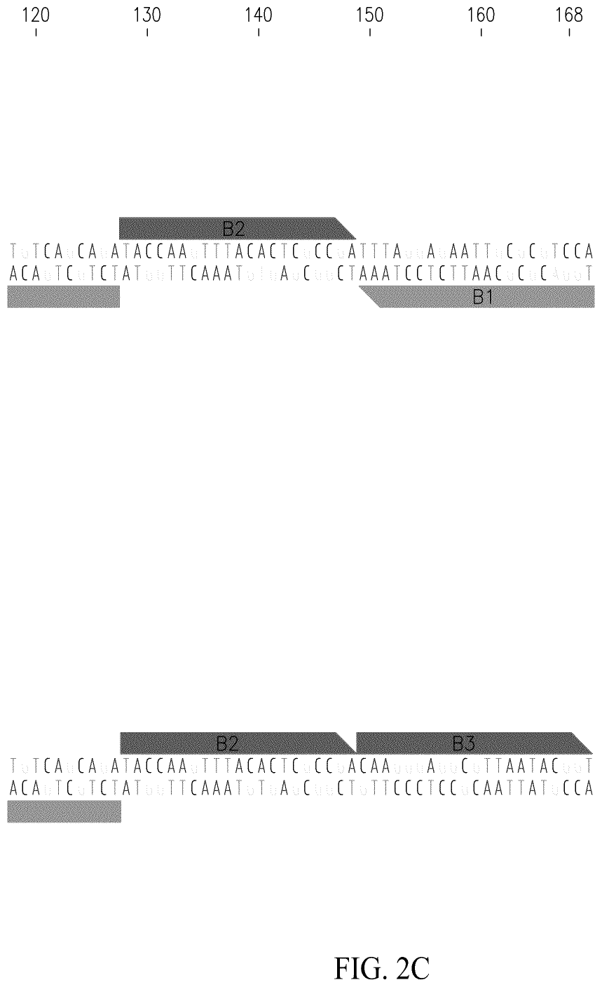

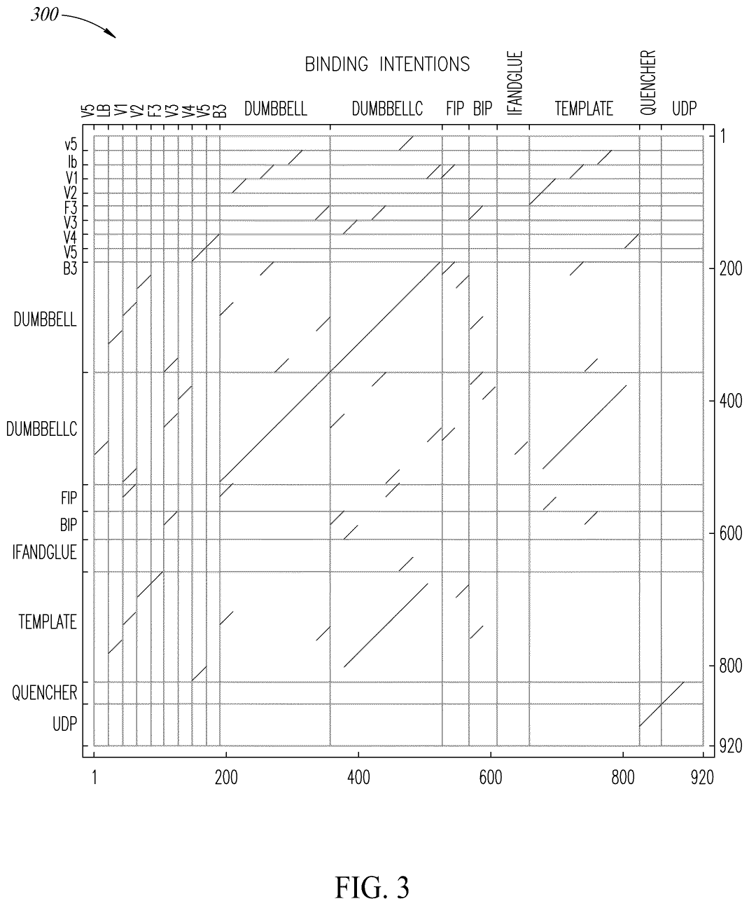

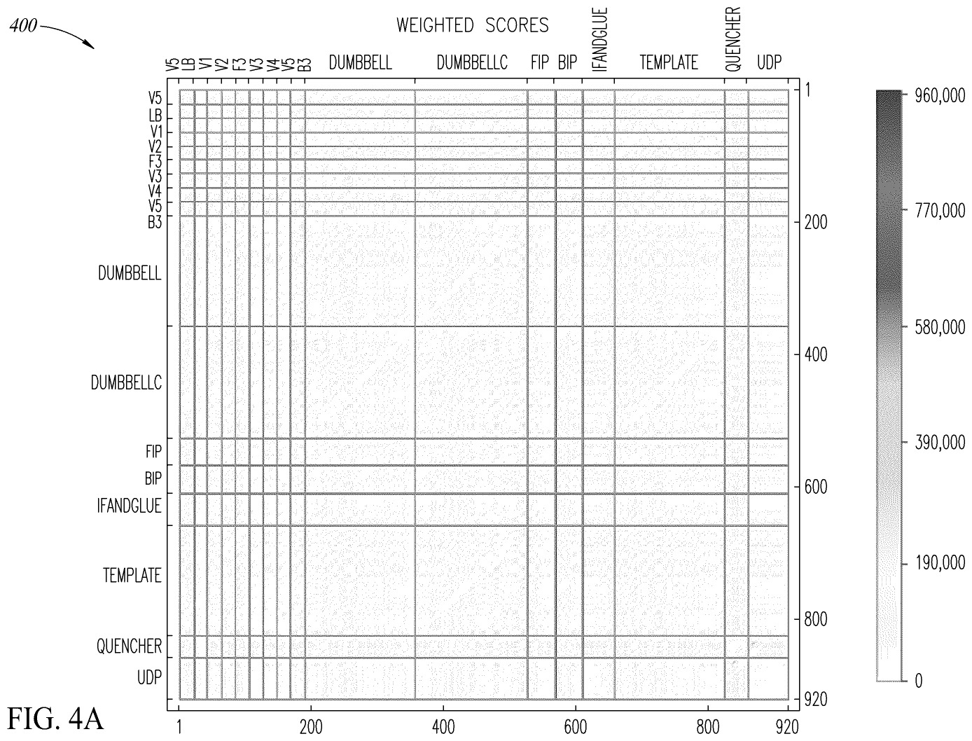

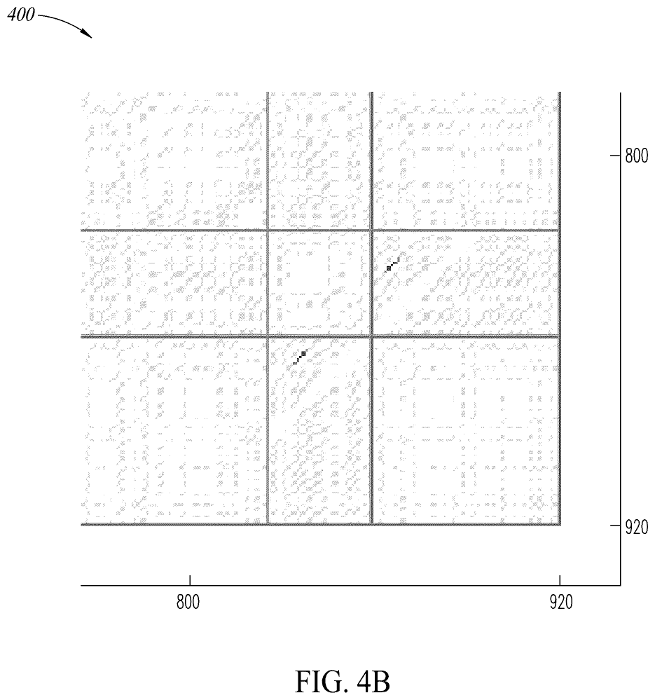

FIGS. 2-4B are illustrations 200, 300 and 400, respectively, of a design example for LAMP internal amplification control (IAC). As shown in FIG. 2, the example includes 229 unique nucleotide bases, 168 of which are mutable, 17 unique strands, and 920 bases in total length. FIG. 3 is a graph 300 showing the binding intent for the LAMP IAC design example, and FIGS. 4A-4B show an example scoring matrix 400 for the design example.

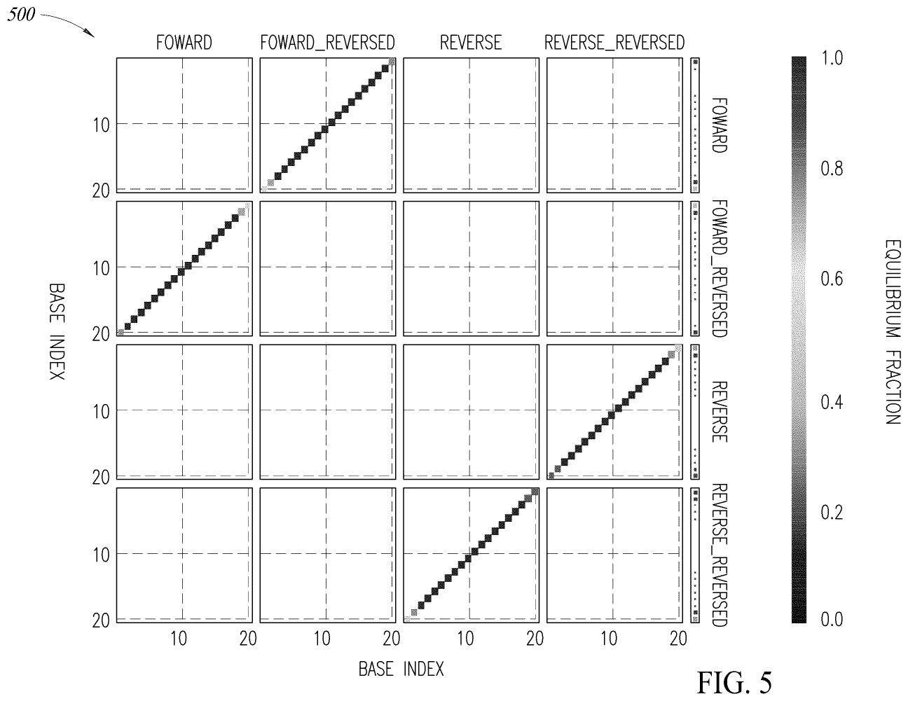

FIG. 5 is an illustration 500 of example computed binding probabilities for a system of two 20 mers (“forward” and “reverse”) and their complements, generated by NUPACK analysis software (available at www.nupack.org). As discussed above, given a nucleotide pair, we see the probability that they will bind with each other. Experimentally faithful algorithms are well-known, which are embodied in tools such as the NUPACK analysis tool. Unfortunately, the algorithms are relatively slow, so it is impractical to run a large number (e.g., millions) of iterates within a reasonable period of time.

The inventor of the present disclosure has recognized that the binding behavior of DNA is most heavily influenced by local effects. Thus, according to embodiments disclosed herein, accurate probabilities may be approximated using myopic neighborhoods, which may be pre-computed and stored in a database for subsequent retrieval. Further, as discussed above, a scoring matrix can be incrementally updated as mutations are made, which provides a linear-time algorithm.

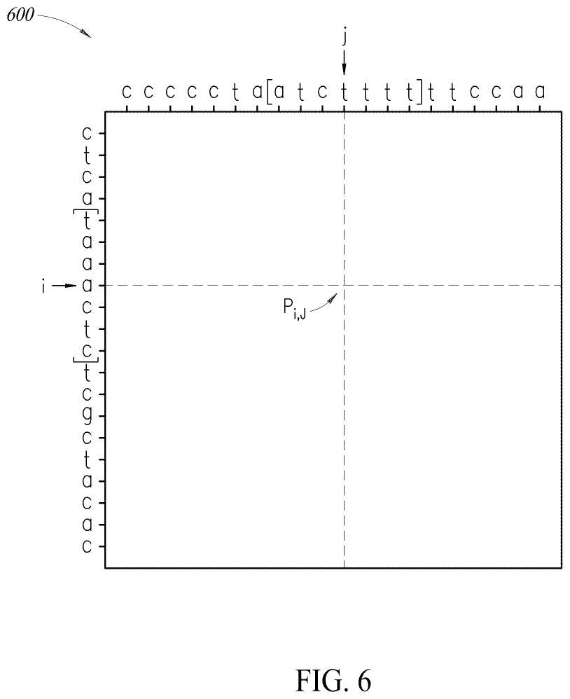

FIG. 6 shows a simplified example matrix 600 that includes elements p_{i,j} that are the precomputed binding probabilities for nucleotides i and j, which probabilities consider the binding behavior only in a limited myopic neighborhood surrounding each nucleotide i and j in their respective strands, as discussed above. In the illustrated example, the neighborhood size k is equal to 7, providing the neighborhood for the nucleotide i of “taaactc,” and providing the neighborhood for the nucleotide j of “atctttt.”

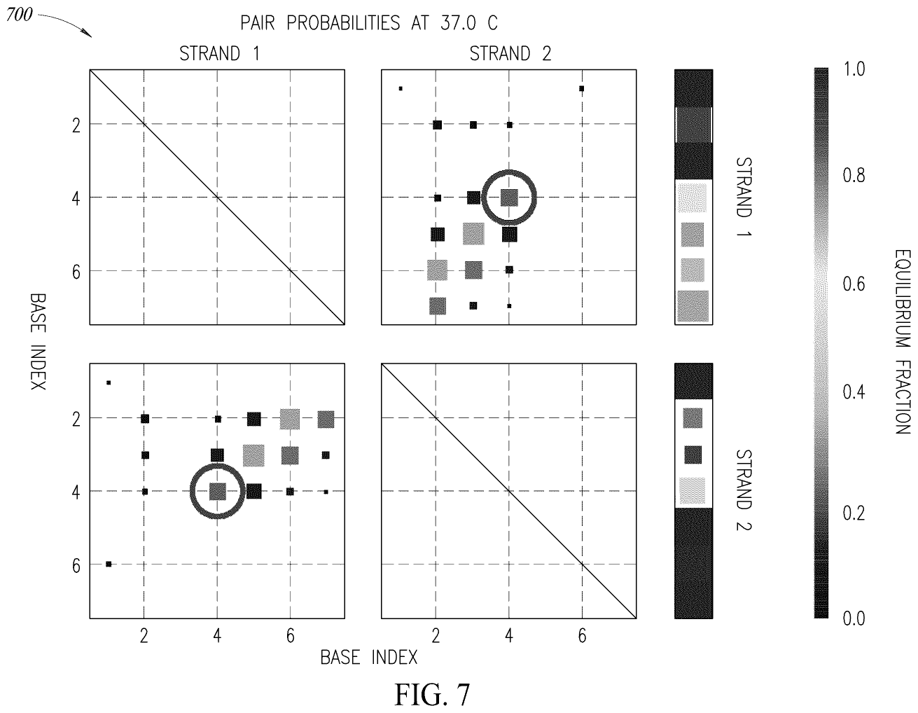

FIG. 7 shows an illustration 700 of a pair-wise central binding probability for T and A in the strands actTttt and taaActc, respectively. The analysis tool (e.g., NUPACK) may be run on a system of two strands as input having lengths that are less than or equal to k. All possible oligos that are less than or equal to k size may be determined, and the binding probabilities may be stored in memory. Thus, the binding probability computation becomes a simple and fast memory lookup in a database, as discussed above.

The neighborhood size k may be selected as a key parameter based on various factors. Generally, larger is better, and odd values are easier than even values due to symmetry, as discussed above. Ultimately, the size of k may be selected based on the desired size of computer memory to be used to store the pre-computed matrices. Further, in at least some implementations, pre-computed databases or matrices may be generated for various conditions (e.g., various combinations of temperatures and salts), and the available database that is “closest” to the conditions of the system being designed may be used.

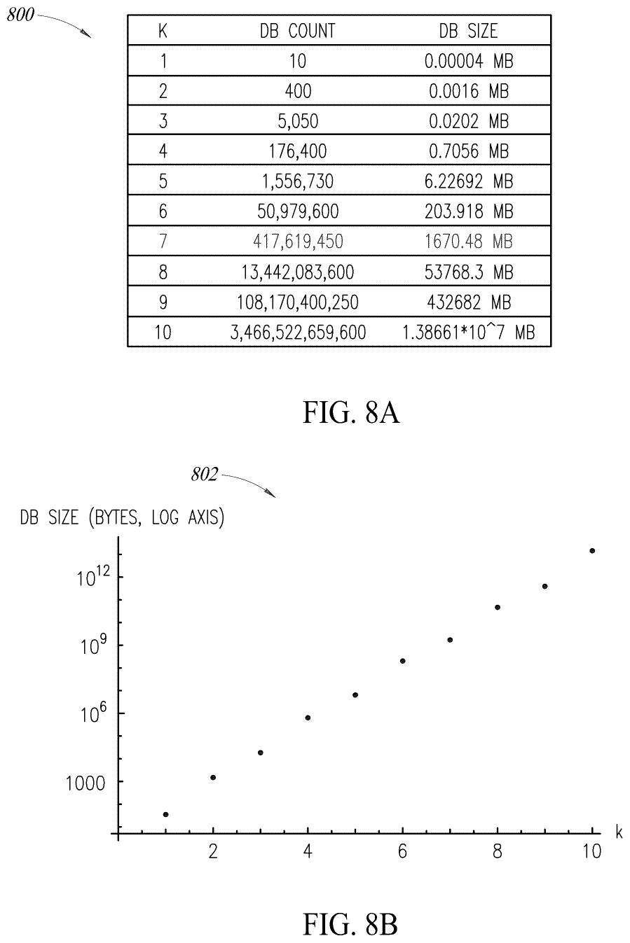

FIG. 8A shows a table 800 of the number of database entries and database size (in MB) for neighborhood sizes between 1 and 10 nucleotides. As shown in a graph 802 of FIG. 8B, the number of runs of the analysis tool, and the size of the database, grows rapidly with increasing neighborhood size. For k=7, the number of runs of the analysis tool is 417,619,450, and the size of the database is 1,670.48 MB.

In at least some implementations, a complication may be long runs of unintended (“bad”) binding that increase the stability of off-design secondary structure. To compensate for this undesirable effect, in at least some implementations an additional scoring weight exponential in bad-binding run-length may be multiplicatively applied to offending nucleotides.

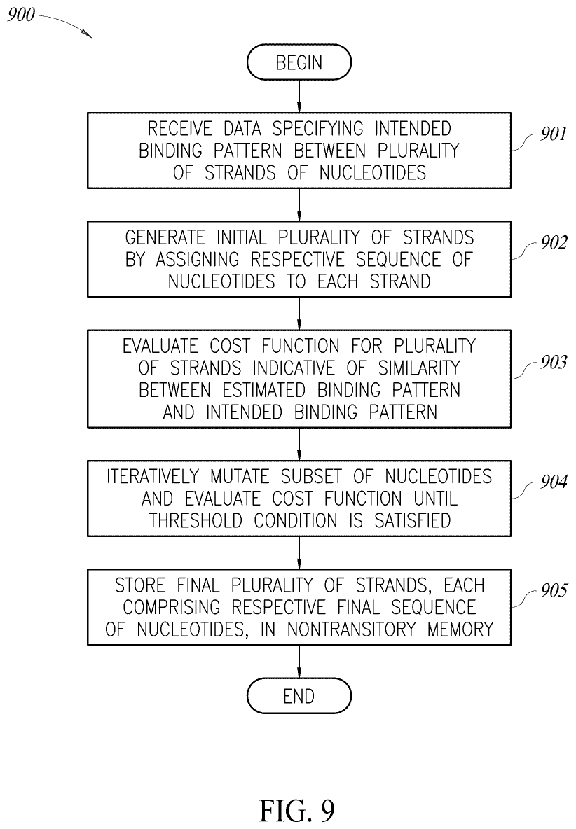

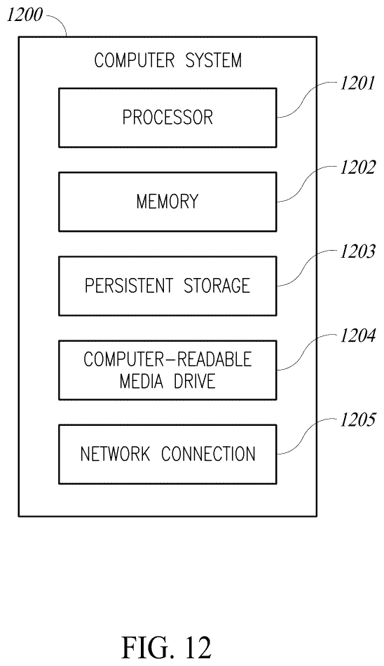

FIG. 9 is a flowchart for a method 900 of performing synthetic nucleic acid sequence design, according to one non-limiting illustrated implementation. The method 900 may be performed by one or more computer systems, such as the example computer system 1200 of FIG. 12 discussed below.

At 901, a processor of the computer system may receive data specifying an intended binding pattern between a plurality of strands of nucleotides (e.g., DNA, RNA). Each of the plurality of strands may include a sequence of nucleotides having a respective length of nucleotides. The intended binding pattern may be specified as binary values, weighted scores, combinations thereof, etc.

At 902, the processor may generate an initial plurality of strands by assigning a respective sequence of nucleotides to each of the plurality of strands. As an example, the at least one processor may randomly initially assign nucleotides (e.g., A, G, C, T) using probability distributions or other criteria.

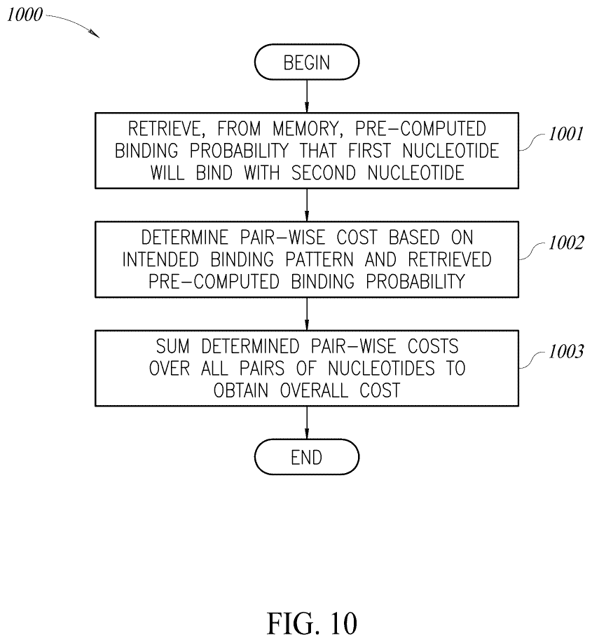

At 903, the processor may evaluate a cost function for the initial plurality of strands. The cost function may be indicative of the similarity between a heuristically estimated binding pattern of the plurality of strands and the intended binding pattern. FIG. 10, discussed below, shows an example method 1000 of evaluating a cost function.

As an example, the pair-wise cost may be determined according to the formula:

c_{i,j}=(t_{i,j}+1)/2−t_{i,j}×p_{i,j}

wherein c_{i,j} is the pair-wise cost for the first nucleotide and the second nucleotide, t_{i,j} is +1 for an intended binding and −1 for an intended non-binding, and p_{i,j} is the pre-computed binding probability. As another example, the pair-wise cost may be determined according to the formula:

c_{i,j}=0 for an intended binding; and

c_{i,j}=p_{i,j} for an intended non-binding,

wherein c_{i,j} is the pair-wise cost for the first nucleotide and the second nucleotide, and p_{i,j} is the pre-computed binding probability. Other cost functions may be used as well.

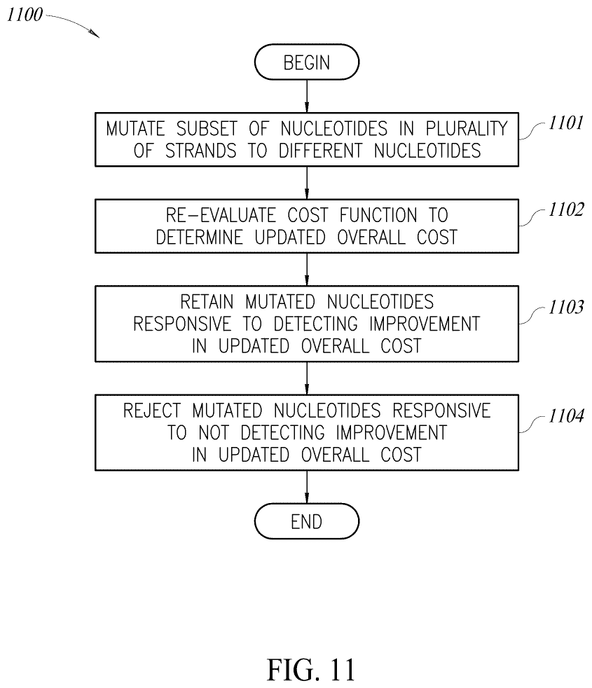

At 904, the processor may iteratively mutate a subset of nucleotides and re-evaluate the cost function until a threshold condition is satisfied. FIG. 11, discussed further below, shows an example method 1100 of mutating nucleotides, re-scoring, and looping until a threshold is satisfied. In at least some implementations, the processor may incrementally evaluate the cost function based on the nucleotides that were mutated relative to the previous iteration, allowing previously calculated scores that are unaffected by the mutations to be retained. The threshold condition may include one or more of an amount of improvement in the overall cost, a number of iterations, an elapsed time, etc.

At 905, the processor may store a final plurality of strands, each comprising a respective final sequence of nucleotides, in the at least one nontransitory memory. The final plurality of strands may then be used for numerous practical applications, as discussed above, including molecular probes for diagnostics, molecular computation, forensics, etc.

FIG. 10 is a flowchart for a method 1000 of evaluating a cost function, according to one non-limiting illustrated implementation. The method 1000 may be performed by one or more computer systems, such as the computer system 1200 of FIG. 12 discussed below. The method may be performed for every pair of nucleotides in every pair of the plurality of strands in a system design, wherein each pair of nucleotides includes a first nucleotide from one of the plurality of strands and a second nucleotide from the same or another of the plurality of strands.

At 1001, the processor retrieves, from at least one nontransitory memory, a pre-computed heuristic binding probability that the first nucleotide will bind with the second nucleotide. The pre-computed binding probability may be based on a model that considers the binding behavior of only a first neighborhood of adjacent nucleotides that includes the first nucleotide and a second neighborhood of adjacent nucleotides that includes the second nucleotide. Each of the first and second neighborhoods has a neighborhood length that is less than or equal to a maximum neighborhood length (e.g., 3, 7, 10, 14, 18, 25). The binding probabilities may be stored in one or more 2D matrices, wherein each of the one or more matrices includes binding probabilities for nucleotides in particular conditions (e.g., temperature and/or salt condition). In at least some implementations, the pre-computed binding probabilities may be retrieved from a database addressable by a linear index that is determinable using data identifying the first and second neighborhoods of nucleotides.

At 1002, the processor determines a pair-wise cost based on the intended binding pattern and the retrieved pre-computed binding probability. At 1003, the processor sums the determined pair-wise costs over all of the pairs of nucleotides to obtain an overall cost. In at least some implementations, the overall cost may be based at least in part on the thermodynamics of binding of the plurality of strands or other criteria.

FIG. 11 is a flowchart for a method 1100 of mutating nucleotides, re-scoring, and looping until a threshold is satisfied, according to one non-limiting illustrated implementation. The method 1100 may be performed by one or more computer systems, such as the computer system 1200 of FIG. 12 discussed below. The method 1100 may be performed iteratively during a nucleic acid sequence design process until a threshold condition is satisfied.

At 1101, the processor mutates a subset of the nucleotides in at least one of the plurality of strands to different nucleotides. As discussed elsewhere herein, the processor may select nucleotides according to a function that is biased toward worst-scoring nucleotides, and/or the number of mutations may be selected according to a probability distribution (e.g., Poisson distribution). The selected nucleotides may be mutated using probability distributions.

At 1102, the processor may re-evaluate the cost function to determine an updated overall cost. At 1103, the processor may retain the mutated nucleotides responsive to detecting an improvement in the updated overall cost relative to a previously-computed overall cost. At 1104, the processor may reject the mutated nucleotides responsive to not detecting an improvement in the updated overall cost relative to a previously-computed overall cost. As discussed above, the process may loop until a specified condition is met.

FIG. 12 is a block diagram showing some of the components typically incorporated in at least some of the computer systems and other devices on which the systems of the present disclosure operate. In various embodiments, these computer systems and other devices 1200 can include one or more server computer systems, cloud computing platforms or virtual machines in other configurations, desktop computer systems, laptop computer systems, tablet computers, netbooks, mobile phones, personal digital assistants, televisions, cameras, automobile computers, electronic media players, physiological sensing devices, wearable computing devices, associated display devices, etc.

In various embodiments, the computer systems and devices 1200 include zero or more of each of the following: a processor 1201 for executing computer programs such as a central processing unit (CPU), graphics processing unit (GPU), tensor processing unit (TPU), neural network processor (NNP), field-programmable gate array (FPGA), complex programmable logic device (CPLD), application-specific integrated circuit (ASIC), or other hardware circuitry; a computer memory 1202 for storing programs and data while they are being used (e.g., volatile memory, non-volatile memory), including the systems and modules discussed herein and associated data, an operating system including a kernel, and device drivers; a persistent storage device 1203, such as a hard drive or flash drive for persistently storing programs and data; a computer-readable media drive 1204, such as a floppy, CD-ROM, or DVD drive, for reading programs and data stored on a computer-readable medium; and a network connection 1205 for connecting the computer system to other computer systems to send and/or receive data wirelessly or via a wired connection, such as via the Internet or another network and its networking hardware, such as switches, routers, repeaters, electrical cables and optical fibers, light emitters and receivers, radio transmitters and receivers, and the like. While computer systems configured as described above are typically used to support the operation of the functionality described herein, those skilled in the art will appreciate that the systems and methods may be implemented using devices of various types and configurations, and having various components.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof In one implementation, the present subject matter may be implemented via Application Specific Integrated Circuits (ASICs). However, those skilled in the art will recognize that the implementations disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

Those of skill in the art will recognize that many of the methods or algorithms set out herein may employ additional acts, may omit some acts, and/or may execute acts in a different order than specified.

In addition, those skilled in the art will appreciate that the mechanisms taught herein are capable of being distributed as a program product in a variety of forms, and that an illustrative implementation applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory.

The various implementations described above can be combined to provide further implementations. These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A system, comprising:
at least one nontransitory memory that stores at least one of processor-executable instructions or data; and
at least one processor communicatively coupled to the at least one nontransitory memory, in operation, the at least one processor:
receives data specifying an intended binding pattern between a plurality of strands of nucleotides, each of the plurality of strands comprising a sequence of nucleotides having a respective length of nucleotides;
generates an initial plurality of strands by assigning a respective sequence of nucleotides to each of the plurality of strands;
evaluates a cost function for the plurality of strands, the cost function indicative of the similarity between an estimated binding pattern of the plurality of strands and the intended binding pattern, wherein evaluating the cost function comprises,
for every pair of nucleotides in every pair of the plurality of strands, and each pair of nucleotides comprising a first nucleotide from one of the plurality of strands and a second nucleotide from another of the plurality of strands,
retrieving, from the at least one nontransitory memory, a pre-computed binding probability that the first nucleotide will bind with the second nucleotide, wherein the pre-computed binding probability is based on a model that considers the binding behavior of only a first neighborhood of adjacent nucleotides that includes the first nucleotide and a second neighborhood of adjacent nucleotides that includes the second nucleotide, wherein each of the first and second neighborhoods has a neighborhood length that is less than or equal to a maximum neighborhood length;
determining a pair-wise cost based on the intended binding pattern and the retrieved pre-computed binding probability;
summing the determined pair-wise costs over all of the pairs of nucleotides to obtain an overall cost;
iteratively, until a threshold condition is satisfied,
mutates a subset of the nucleotides in at least one of the plurality of strands to different nucleotides;
re-evaluates the cost function to determine an updated overall cost;
retains the mutated nucleotides responsive to detecting an improvement in the updated overall cost relative to a previously-computed overall cost; and
rejects the mutated nucleotides responsive to not detecting an improvement in the updated overall cost relative to a previously-computed overall cost; and
stores a final plurality of strands, each comprising a respective final sequence of nucleotides, in the at least one nontransitory memory.

2. The system of claim 1 wherein the at least one processor generates the initial plurality of strands by assigning a respective sequence of nucleotides to each of the plurality of strands using at least one probability distribution.

3. The system of claim 1 wherein, to evaluate the cost function to determine an updated overall cost, the at least one processor incrementally evaluates the cost function based on the nucleotides that were mutated relative to the previous iteration.

4. The system of claim 1 wherein the overall cost is based at least in part on the thermodynamics of binding of the plurality of strands.

5. The system of claim 1 wherein the at least one processor mutates a subset of the nucleotides based on at least one probability distribution.

6. The system of claim 1 wherein the threshold condition comprises one or more of an amount of improvement in the overall cost, a number of iterations, or an elapsed time.

7. The system of claim 1 wherein the maximum neighborhood length is equal to seven.

8. The system of claim 1 wherein the maximum neighborhood length is greater than or equal to seven.

9. The system of claim 1 wherein the maximum neighborhood length is an odd integer.

10. The system of claim 1 wherein the maximum neighborhood length is an odd integer, and the first and second nucleotides are the center nucleotides in the first and second neighborhoods, respectively.

11. The system of claim 1 wherein the maximum neighborhood length is an even integer, and the first and second nucleotides are nucleotides just to the left or right of the center of the first and second neighborhoods, respectively.

12. The system of claim 1 wherein the pair-wise cost is determined according to the formula:
c_{i,j}=(t_{i,j}+1)/2−t_{i,j}×p_{i,j}
wherein c_{i,j} is the pair-wise cost for the first nucleotide and the second nucleotide, t_{i,j} is +1 for an intended binding and −1 for an intended non-binding, and p_{i,j} is the pre-computed binding probability.

13. The system of claim 1 wherein the pair-wise cost is determined according to the formula:
c_{i,j}=0 for an intended binding; and
c_{i,j}=p_{i,j} for an intended non-binding,
wherein c_{i,j} is the pair-wise cost for the first nucleotide and the second nucleotide, and p_{i,j} is the pre-computed binding probability.

14. The system of claim 1 wherein each of the plurality of strands comprise a sequence of four nucleotides.

15. The system of claim 1 wherein the at least one processor receives data specifying at least one of a temperature condition or a salt condition, and wherein the retrieved pre-computed binding probabilities are based at least part on the temperature condition or salt condition.

16. The system of claim 1 wherein the pre-computed binding probabilities are retrieved from a database addressable by a linear index that is determinable using data identifying the first and second neighborhoods.

17. A processor-implemented method, comprising:
receiving data specifying an intended binding pattern between a plurality of strands of nucleotides, each of the plurality of strands comprising a sequence of nucleotides having a respective length of nucleotides;
generating an initial plurality of strands by assigning a respective sequence of nucleotides to each of the plurality of strands;
evaluating a cost function for the plurality of strands, the cost function indicative of the similarity between an estimated binding pattern of the plurality of strands and the intended binding pattern, wherein evaluating the cost function comprises,
for every pair of nucleotides in every pair of the plurality of strands, and each pair of nucleotides comprising a first nucleotide from one of the plurality of strands and a second nucleotide from another of the plurality of strands,
retrieving, from at least one nontransitory memory, a pre-computed binding probability that the first nucleotide will bind with the second nucleotide, wherein the pre-computed binding probability is based on a model that considers the binding behavior of only a first neighborhood of adjacent nucleotides that includes the first nucleotide and a second neighborhood of adjacent nucleotides that includes the second nucleotide, wherein each of the first and second neighborhoods has a neighborhood length that is less than or equal to a maximum neighborhood length;
determining a pair-wise cost based on the intended binding pattern and the retrieved pre-computed binding probability;
summing the determined pair-wise costs over all of the pairs of nucleotides to obtain an overall cost;
iteratively, until a threshold condition is satisfied, mutating a subset of the nucleotides in at least one of the plurality of strands to different nucleotides;
re-evaluating the cost function to determine an updated overall cost;
retaining the mutated nucleotides responsive to detecting an improvement in the updated overall cost relative to a previously-computed overall cost; and
rejecting the mutated nucleotides responsive to not detecting an improvement in the updated overall cost relative to a previously-computed overall cost; and
storing a final plurality of strands, each comprising a respective final sequence of nucleotides, in the at least one nontransitory memory.

18. The method of claim 17 wherein generating the initial plurality of strands comprises generating the initial plurality of strands by assigning a respective sequence of nucleotides to each of the plurality of strands using at least one probability distribution.

19. The method of claim 17 wherein evaluating the cost function to determine an updated overall cost comprises incrementally evaluating the cost function based on the nucleotides that were mutated relative to the previous iteration.

20. The method of claim 17 wherein the overall cost is based at least in part on the thermodynamics of binding of the plurality of strands.

21. The method of claim 17 wherein mutating a subset of the nucleotides comprises mutating a subset of the nucleotides based on at least one probability distribution.

22. The method of claim 17 wherein the threshold condition comprises one or more of an amount of improvement in the overall cost, a number of iterations, or an elapsed time.

23. The method of claim 17 wherein the maximum neighborhood length is greater than or equal to seven.

24. The method of claim 17 wherein the maximum neighborhood length is an odd integer, and the first and second nucleotides are the center nucleotides in the first and second neighborhoods, respectively.

25. The method of claim 17 wherein the pair-wise cost is determined according to the formula:
c_{i,j}=0 for an intended binding; and
c_{i,j}=p_{i,j} for an intended non-binding,
wherein c_{i,j} is the pair-wise cost for the first nucleotide and the second nucleotide, and p_{i,j} is the pre-computed binding probability.

26. The method of claim 17 wherein the at least one processor receives data specifying at least one of a temperature condition or a salt condition, and wherein the retrieved pre-computed binding probabilities are based at least part on the temperature condition or salt condition.

27. The method of claim 17 wherein the pre-computed binding probabilities are retrieved from a database addressable by a linear index that is determinable using data identifying the first and second neighborhoods.

28. A non-transitory computer memory that stores at least one of instructions or data that, when executed by at least one processor, cause the at least one processor to perform operations, the operations comprising:
receiving data specifying an intended binding pattern between a plurality of strands of nucleotides, each of the plurality of strands comprising a sequence of nucleotides having a respective length of nucleotides;
generating an initial plurality of strands by assigning a respective sequence of nucleotides to each of the plurality of strands;
evaluating a cost function for the plurality of strands, the cost function indicative of the similarity between an estimated binding pattern of the plurality of strands and the intended binding pattern, wherein evaluating the cost function comprises,
for every pair of nucleotides in every pair of the plurality of strands, and each pair of nucleotides comprising a first nucleotide from one of the plurality of strands and a second nucleotide from another of the plurality of strands,
retrieving, from at least one nontransitory memory, a pre-computed binding probability that the first nucleotide will bind with the second nucleotide, wherein the pre-computed binding probability is based on a model that considers the binding behavior of only a first neighborhood of adjacent nucleotides that includes the first nucleotide and a second neighborhood of adjacent nucleotides that includes the second nucleotide, wherein each of the first and second neighborhoods has a neighborhood length that is less than or equal to a maximum neighborhood length;
determining a pair-wise cost based on the intended binding pattern and the retrieved pre-computed binding probability;
summing the determined pair-wise costs over all of the pairs of nucleotides to obtain an overall cost;
iteratively, until a threshold condition is satisfied,
mutating a subset of the nucleotides in at least one of the plurality of strands to different nucleotides;
re-evaluating the cost function to determine an updated overall cost;
retaining the mutated nucleotides responsive to detecting an improvement in the updated overall cost relative to a previously-computed overall cost; and
rejecting the mutated nucleotides responsive to not detecting an improvement in the updated overall cost relative to a previously-computed overall cost; and
storing a final plurality of strands, each comprising a respective final sequence of nucleotides, in the at least one nontransitory memory.


SEQUENCE LISTING (22 sequences)

SEQ ID NO: 1 - DNA, 21 bp, synthetic construct
  source 1..21: mol_type = other DNA
caagggaggcgttaatacggt

SEQ ID NO: 2 - DNA, 21 bp, synthetic construct
  source 1..21: mol_type = other DNA
accgtattaacgcctcccttg

SEQ ID NO: 3 - DNA, 42 bp, synthetic construct
  source 1..42: mol_type = other DNA
tttaggagaattgcgcgtccataccaagtttacactcgccga

SEQ ID NO: 4 - DNA, 42 bp, synthetic construct
  source 1..42: mol_type = other DNA
tcggcgagtgtaaacttggtatggacgcgcaattctcctaaa

SEQ ID NO: 5 - DNA, 168 bp, synthetic construct
  source 1..168: mol_type = other DNA
gtgcccgaggtttcatctcaatggaaagatcgtcgccctctatcccgcacacagagtaagaaattgagatgaaacctcgggcactggacgcgcaattctcctaaatatgtgagccatgtcagcagataccaagtttacactcgccgatttaggagaattgcgcgtcca

SEQ ID NO: 6 - DNA, 168 bp, synthetic construct
  source 1..168: mol_type = other DNA
tggacgcgcaattctcctaaatcggcgagtgtaaacttggtatctgctgacatggctcacatatttaggagaattgcgcgtccagtccccgaggtttcatctcaatttcttactctgtgtgcgggatagagggcgacgatctttccattgagatgaaacctcgggcac

SEQ ID NO: 7 - DNA, 21 bp, synthetic construct
  source 1..21: mol_type = other DNA
tgctttgcagcctagcattca

SEQ ID NO: 8 - DNA, 21 bp, synthetic construct
  source 1..21: mol_type = other DNA
tgaatgctaggctgcaaagca

SEQ ID NO: 9 - DNA, 42 bp, synthetic construct
  source 1..42: mol_type = other DNA
ttgagatgaaacctcgggcactagagggcgacgatctttcca

SEQ ID NO: 10 - DNA, 42 bp, synthetic construct
  source 1..42: mol_type = other DNA
tggaaagatcgtcgccctctagtgcccgaggtttcatctcaa

SEQ ID NO: 11 - DNA, 21 bp, synthetic construct
  source 1..21: mol_type = other DNA
tctgctgacatggctcacata

SEQ ID NO: 12 - DNA, 21 bp, synthetic construct
  source 1..21: mol_type = other DNA
tatgtgagccatgtcagcaga

SEQ ID NO: 13 - DNA, 48 bp, synthetic construct
  source 1..48: mol_type = other DNA
accacacctaccaccactaataactaatcccgcacacagagtaagaaa

SEQ ID NO: 14 - DNA, 48 bp, synthetic construct
  source 1..48: mol_type = other DNA
tttcttactctgtgtgcgggattagttattagtggtggtaggtgtggt

SEQ ID NO: 15 - DNA, 34 bp, synthetic construct
  source 1..34: mol_type = other DNA
ctgtcgtaggtggttgggttcggaagtggtcagg

SEQ ID NO: 16 - DNA, 34 bp, synthetic construct
  source 1..34: mol_type = other DNA
cctgaccacttccgaacccaaccacctacgacag

SEQ ID NO: 17 - DNA, 168 bp, synthetic construct
  source 1..168: mol_type = other DNA
tgaatgctaggctgcaaagcatggaaagatcgtcgccctctatcccgcacacagagtaagaaattgagatgaaacctcgggcactggacgcgcaattctcctaaatatgtgagccatgtcagcagataccaagtttacactcgccgacaagggaggcgttaatacggt

SEQ ID NO: 18 - DNA, 168 bp, synthetic construct
  source 1..168: mol_type = other DNA
accgtattaacgcctcccttgtcggcgagtgtaaacttggtatctgctgacatggctcacatatttaggagaattgcgcgtccagtgcccgaggtttcatctcaatttcttactctgtgtgcgggatagagggcgacgatctttccatgctttgcagcctagcattca

SEQ ID NO: 19 - DNA, 61 bp, synthetic construct
  source 1..61: mol_type = other DNA
cctgaccacttccgaacccaaccacctacgacagaccacacctaccaccactaataactaa

SEQ ID NO: 20 - DNA, 61 bp, synthetic construct
  source 1..61: mol_type = other DNA
ttagttattagtggtggtaggtgtggtctgtcgtaggtggttgggttcggaagtggtcagg

SEQ ID NO: 21 - DNA, 20 bp, synthetic construct
  source 1..20: mol_type = other DNA
ccccctaatcttttttccaa

SEQ ID NO: 22 - DNA, 20 bp, synthetic construct
  source 1..20: mol_type = other DNA
ctcataaactctcgctacac